(19) United States
(12) Patent Application Publication
(10) Pub. No.: US 2016/0354307 A1
(43) Pub. Date: Dec. 8, 2016

(54) ANTIMICROBIAL COMPOSITION AND METHODS OF USE

(71) Applicants: Carl Hilliard, San Diego, CA (US); William R. Cast, Fort Wayne, IN (US)

(72) Inventors: Carl Hilliard, San Diego, CA (US); William R. Cast, Fort Wayne, IN (US)

(21) Appl. No.: 15/231,236

(22) Filed: Aug. 8, 2016

Related U.S. Application Data
(63) Continuation-in-part of application No. 14/716,589, filed on May 19, 2015.
(60) Provisional application No. 62/000,403, filed on May 19, 2014.
(60) Provisional application No. 62/201,963, filed on Aug. 6, 2015.

(51) Int. Cl. A61K 9/00; A61L 12/14; A61K 31/695

(52) U.S. Cl. CPC A61K 9/0048; A61K 31/695; A61K 9/0051; A61L 12/143

(57) ABSTRACT

An antimicrobial composition and methods of use are provided. The antimicrobial composition includes a water-soluble antimicrobial organosilane ammonium compound and a sugar. The composition may be in liquid, foam, ointment or gel form. The composition may include anti-inflammatory medications, alcohol and/or steroids. The antimicrobial composition may be used to treat eye infections in humans and animals. The antimicrobial composition may be applied directly to the surface of the eye. The antimicrobial composition may further be used to treat ocular related articles, including contact lenses, contact cases, instruments, pads and the like. The antimicrobial composition may be applied directly to the surface of the ocular related article during and/or after manufacture.

### CROSS REFERENCE TO RELATED APPLICATION[S]

This application claims priority to earlier U.S. Provisional Patent Application entitled “PRODUCT AND DELIVERY SYSTEM FOR ANTIMICROBIAL TREATMENT OF INFECTIONS OF THE EYE AND OF PATHOGENS CONTAMINATING OCULAR DEVICES AND METHODS OF USE,” Ser. No. 62/201,693, filed Aug. 6, 2015; and further, this application is a continuation-in-part of the earlier U.S. Utility Patent Application entitled “PRODUCT AND METHOD FOR TREATMENT OF A BIOFILM, INCLUDING CONTROL OF SUBSTRATE COLONIZATION AND TREATMENT OF INFECTION,” Ser. No. 14/716,589, filed May 19, 2015, which claims priority to earlier U.S. Provisional Patent Application entitled “ANTIMICROBIAL POLYMER PRODUCTS AND DELIVERY SYSTEM FOR INFECTION CONTROL AND METHOD OF USING THE SAME,” Ser. No. 62/200,403, filed May 19, 2014, the disclosures of which are hereby incorporated entirely herein by reference.

### BACKGROUND

Technical Field

The invention relates generally to antimicrobial compositions and methods of use, particularly to ocular antimicrobial compositions for treatment of eye infections and treatment of ocular related articles.

State of the Art

Treatment of corneal disease is complicated by the difficulty in diagnosis, at both the clinical and laboratory level, of the pathogen(s) causing the infection. These pathogens take days or even weeks to culture and to grow during which time significant irreversible damage may well occur to the infected eye. Even after diagnosis, the medications available for treatment are limited, to wit: the antibiotics used in ophthalmology do not have significant activity across groups of potential pathogens and there is a lack of potent fungicidal agents and poor ocular penetration of existing agents. The paucity of effective drugs is further diminished by a growing number of multi-drug resistant organisms

Keratitis is a general term meaning any inflammation of the cornea (the clear, round dome covering the eye's iris and pupil). The risk factors for keratitis include diabetes, AIDS, trauma to the eye, contact lens wear, contaminated lens cases and solutions, topical steroid use, use of traditional eye remedies, contaminated medications and make-up and ocular surface disorders. There are approximately thirty-six million lens users in the United States with 12,000 to 15,000 cases of keratitis each year. Overnight wear of contact lenses is the overwhelming risk factor.

Fungal keratitis is notoriously difficult to treat because of poor corneal penetration of antifungal agents. The only commercially available agent is natacyn and other agents needed to treat some of the seventy varieties of fungus must be compounded. Fungal keratitis has also been observed after LASIK procedures and associated denervation, as well as after corneal transplant in which the patient's corneal nerves are compromised, foreign material is present in the form of sutures and there is a concomitant use of a topical corticosteroid. The incidence of fungal keratitis has increased due to frequent use of topical corticosteroids along with antibacterial agents.

Bacterial keratitis causes pain, reduced vision, light sensitivity and tearing or discharge from the eye, and can also cause blindness. This disease is characterized by rapid progression. Destruction of the cornea may be complete within 24-48 hours with some of the more virulent bacteria. The characteristics of this disease are corneal ulceration, stromal abscess formation, surrounding corneal edema, and anterior segment inflammation. Bacterial keratitis is a common problem in contact lens use and refractive corneal surgery.

Polymicrobial infection is not uncommon and may be caused by combinations of viruses, bacteria and fungi. These multi-pathogen infections have been found in a third of cases, the majority due to multiple bacterial species. Twenty percent of positive cultures from cases with fungal keratitis were co-infected with bacteria. The risk of polymicrobial infection was approximately three times greater with Candida yeast fungi than with infection with filamentous fungi. This finding suggests that the bacterial are protected within the biofilm produced by the Candida fungi (the most common cause of fungal infection) and may contribute to the generally poor prognosis for fungal keratitis.

Conjunctivitis may be caused by a bacterial or viral infection, allergy, environmental irritants, contact lens products, eye drops, or eye ointments. Conjunctivitis causes swelling, itching, burning, and redness of the conjunctiva, the protective membrane that lines the eyelids and covers exposed areas of the sclera, or white of the eye. Conjunctivitis can spread from one person to another and affects millions of Americans at any given time. Some forms of conjunctivitis require medical treatment. If treatment is delayed, the infection may worsen and cause corneal inflammation and a loss of vision. Corneal infections are the most serious complication of contact lens wearers.

Compliance is a factor in inability of a patient/family to administer the medication required to treat many eye infections. Enough medication is required to kill the infection and at the same time be tolerated by the eye. One difficulty in topical administration of antibiotics is that they are rapidly cleared from the pre-corneal area by tear drainage and the immediate effect of blinking. Thus, most antibiotics must be administered frequently with application rates up to hourly and through the night, and in the case of fungal disease, perhaps for weeks of duration.

It was believed for many years that bacteria, unlike eukaryotic organisms, behaved as self-sufficient individuals and maintained a strictly unicellular life-style in planktonic form. During infections, bacterial mass was considered nothing more than the sum of these individuals. Our perception of bacteria as unicellular life-style was deeply rooted in the pure culture paradigm. Pure cultures were used to establish microbial causes of disease, and growth in liquid media ensured that all cells were exposed to similar conditions and behaved in the same manner. As a result, most of the measures to control pathogenic bacteria (e.g., vaccines and antimicrobial agents) have been developed based on knowledge of bacteria grown as planktonic cells. However, pure-culture planktonic growth of bacteria rarely exists in natural environments. In fact, bacteria in Nature largely reside in a complex and dynamic surface-associated community called a biofilm. It is now known that over 99% of bacteria life forms s with as few as 1% living in planktonic form.

Biofilms are generally defined as a community of sessile microbes held together by a polymeric extracellular matrix, adherent to a surface, interface or to other cells that are phenotypically distinct from their planktonic counterparts. Members of a biofilm community, which can be of the same or multiple species, show varying stages of differentiation and exchange information, metabolites, and genes with each other. As a result, members of the biofilm community are in a diversity of physiologies influenced by the unequal sharing of nutrients and metabolic byproducts, which results in subpopulations subjected to differing environmental stresses and having wit4i increased tolerance to antimicrobials and environmental stresses, the host immune system, and predatory microorganisms. Biofilm cell communities are more resistant to antibiotic and antifungal drugs than planktonic cells. Contributing factors include biofilm structural complexity, presence of extracellular matrix (ECM), metabolic heterogeneity intrinsic to biofilms, and biofilm-associated up-regulation of efflux pump genes.

Recent advances in medical biofilm research have led to an understanding that biofilms are responsible for a broad spectrum of microbial infections in the human or animal hosts and represent the prevalent form of bacterial life for tissue colonization, and they have been observed on the capsule, and in the corneal stroma.

The growing problem of antibiotic resistance is well documented in the CDC publication Antibiotic Resistance Threats in the United State, 2013. Candida is singled out in this report because this dangerous fungus is showing increasing resistance to the drugs available for treatment. With the already daunting course an ocular fungal infection already poses due to the paucity of anti-fungals that penetrate the cornea poorly, a drug resistant Candida presents a global threat to corneal health. The story is much the same with respect to other drugs used to treat eye infections: Over 30% of isolates from corneal infections were not sensitive to ciprofloxacin in India, and moxifloxacin and gatifloxacin are not reliable treatments for MRSA. Approximately 85% of MRSA strains are resistant to moxifloxacin and gatifloxacin. Resistance to fluoroquinolones is increasing.

Less well understood is a biofilm defense called antibiotic tolerance. Because of this defense “we actually never had antibiotics capable of eradicating an infection.” Lewis (2012), Persister Cells: Molecular Mechanisms Related to Antibiotic Tolerance, p. 121. A small number of cells (persisters) in a biofilm are phenotypically resistant to sudden exposure to stress brought on by high doses of antibiotics and also phagocytosis by microphages. Once an antibiotic concentration drops, surviving persisters re-establish the population, causing a relapsing chronic infection. Whether persistence and resistance represent complementary or alternative adaptions is unclear, although recent research indicates that they come from separate phenomena. Tolerance allows a population of cells to linger at the site during the decrease of antibiotic concentration which increases the probability of acquiring resistance.

The mechanism of the formation of persister cells has only recently been studied and begun to be understood. “Only a few years ago the molecular basis of persistence was still obscure. Although many genes were known to influence persister formation, they seemed so disparate and general that predicting persistence solely from genomic data would have appeared impossible.” Vogwill, et. al. (2016)J. Evol. Biol. dcl: 10.111/jeb. 12864, p. 1. “The main focus of research in antimicrobials has been on antibiotic resistance, and the basic starting experiment is to test a clinical isolate for its ability to grow in the presence of elevated levels of different antibiotics.” Persister cells are missed by this test. Lewis (2012), Persister Cells, p. 124.

Several models theorize how persister cells escape destruction. The three-dimensional organization of the biofilm causes gradients of oxygen, pH, and nutrients, resulting in the development of different microniches, or microbial microenvironments. The cell's individual physiological adaptations to these microniches results in physiological heterogeneity. Cells near the surface of the biofilm will be exposed to more nutrients, such as salts, amino acids, proteins, sugars and oxygen and are therefore more metabolically active, while cells in the deep regions will be less active or even dormant. This heterogeneity results in a range of responses to antimicrobial agents, with metabolically active cells at the surface being rapidly killed while more internal, dormant cells are comparatively unaffected. Some theories postulate that persister cells adopt a low metabolic state or dormancy and thus become highly resistant to antibiotics. An experiment with ciprofloxacin indicated that a biofilm response was the stress release of the TisB peptide which binds to the membrane of the persister cell causing a metabolic shut down, that blocks antibiotic targets, and ensures multidrug tolerance for the surviving persisters. Another possible route to the formation of persister cells is stochastic production of a few persister cells in each generation of cells that would seem to provide evolutionary protection should the vast majority be destroyed

In some instances, the medications available for treatment are limited because the available antibiotics do not have significant activity across groups of potential pathogens and there is a lack of potent fungicidal agents. “[A]ntimicrobial drugs that specifically target biofilm-associated infections are needed.” CDC, Vol. 10, Number 1 “Fungal Biofilms and Drug Resistance. It is apparent that there is a critical need to find and identify molecules that can overcome both antibiotic resistance and tolerance and can completely destroy biofilms and persister cells.

Biofilm formation also imposes a limitation on the uses and design of ocular devices, such as intraocular lenses, posterior contact lenses, scleral buckles, conjunctival plugs, lacrimal intubation devices and orbital implants. As the evidence for the involvement of microbial biofilms in many ocular infections has become compelling. Biofilm formation begins with a transition from the planktonic form to its genetically distinct sessile state (Colonization). Developing new strategies to prevent colonization has become a priority. One way to reduce ocular related article surface contamination is to sterilize the contact lenses, intraocular lens and lens case. Products used to clean and disinfect contact lenses use heat, subsonic agitation or UV disinfection systems with cleaning solutions that include enzymes or hydrogen peroxides. These systems are intended to remove contamination on the lens. However, it has been found that fungi resist disinfection by contact lens solutions wherein they readily form biofilms. Biofilms have that cause endophthalmitis have also been found on intraocular cataract lenses and contact lenses. Contact lens cases have also been linked to microbial keratitis. Such cases provide an environment that is nutritive and protective of microorganisms that form biofilms. Some disinfectants have been shown to select for resistant antimicrobial strains, e.g., methicillin resistant Staphylococcus aureus (MRSA). MRSA/MRSE and mycobacterial infection in contact lens wear are rare but have devastating effect. Community-associated MRSA is an evolving ocular pathogen most often found in hospital patients. Finally, disinfectants are washed off and must be replaced daily. The repeated use of disinfectants that are sent down the drain poses an environmental problem.

Compliance with safety measures involving contact lens care products is a daunting problem. Patients are not compliant even though they believe and intend otherwise. Contact lens users have a tendency to re-use or top off cleaning solutions. Tap water is often used to rinse lenses or contact lens storage cases instead of sterile water. The recommendation that contact lens cases be thoroughly cleaned and air dried and then replaced every three months is routinely ignored. These non-compliance tendencies raise the risk of contact lens-related eye infections.

To address the exhaustion of biocides to the surrounding environment, a class of water-soluble antimicrobial polymers, Contact-Active Biocidal (CAB) was developed to provide a non-toxic, non-leaching surface covering for walls and counters. CAB products can be bonded to most surfaces, both porous and non-porous, for an extended period of time. As such, the CAB products provide an invisible, microbiostatic coating to prevent Colonization by reducing the number of planktonic single cell microbes that attach to the surface below the number required to form a biofilm. The CAB products are typically offered in liquid form and may be applied to desired surfaces after disinfecting the surface of a wall or counter or through a washing machine rinse cycle. Once the CAB product is applied, the compound reduces the number of new microbes that are able to attach to the surface by creating a semi-permanent coating that partially covers the surface and physically kills microorganisms on contact.

The effective life of the CAB product, however, is relatively short. Moreover, once applied, it is difficult to determine at what time the biological activity becomes diminished and the CAB is no longer maintaining a disinfected surface. An undisclosed problem is a CAB that is not regularly cleaned can be expected to fill with dust and debris which works counter to its claimed purpose. Most CAB products used as coverings for ocular related article surfaces have not been a commercial success.

There is a desperate need in medicine for newer compounds with novel mechanisms of action, greater antimicrobial activity and less cytotoxicity. Therefore, it is desired to provide a composition that address the above concerns, namely, providing an effective substitute for antibiotics and antifungals in treating eye infections; better antifungal agents that work rapidly, penetrate more efficiently into ocular tissues and have fewer medical failures, protecting against infection inadvertently delivered by contact lenses, lens cases and contaminated fluids that are used to disinfect or treat the eye; reducing use of toxic compounds that pollute the environment and reduce the likelihood of microbial development of antibiotic resistance in biofilms.

### DISCLOSURE OF EMBODIEMENTS OF THE INVENTION

The present disclosure relates to broad spectrum antimicrobial compositions, and, in particular, to antimicrobial compositions comprising organosilanes including 3-(trihydroxysilyl)propyldimethyloctadecyl ammonium chloride and methods of use for treating ocular infections; destruction and removal of biofilms and inhibiting the formation of biofilms on eyes, lenses or other devices to be placed in the eye and in lens cases or other containers or repositories for such lenses or devices. These compositions combine both antibacterial and antifungal properties and accordingly are particularly useful when rapid intense topical therapy is required before identification of the pathogen causing the infection can be made or when dealing with a polymicrobial infection.

The antimicrobial compositions inactivate, disrupt and destroy pathogens that cause, inter alia: corneal inflammation, endophthalmitis, anterior segment infection and inflammation, keratitis, scleral buckle infection, corneal ulceration, stromal abscess formation, lacrimal system infections, periorbital infections and infections in the corneal stroma (any of which can cause loss of vision and blindness), and methods of using the same. Use of such compositions is intended by direct application to the eye, to cure eye infections; inhibit re-infection; also to disinfect and create an antimicrobial barrier against the population of infectious microorganisms on ocular related articles, such as intraocular cataract lenses, contact lenses and other devices to be placed or used in or on the eye; and disinfect and provide a barrier against re-infection of lens cases and similar storage devices. The disclosed antimicrobial compositions may be placed topically onto the conjunctiva and cornea. In some embodiments, the organosilane is 3-(trihydroxysilyl) quaternary ammonium chloride. In a preferred embodiment, the formulation includes a pharmaceutically acceptable topical carrier and a delivery system, which is applied directly into the infected eye of an animal or human. The rapid bonding quality of the composition inhibits premature clearing by tearing and permits low concentration of the active ingredient at an effective level of minimal irritation to tissues.

The antimicrobial compositions described herein may be used for treatment of ocular articles relating to the eye, inter alia contact lenses, lens cases, protective shields that contact the sclera or cornea, suture material, for some embodiments the delivery system is a pad, cloth or other material treated with the antimicrobial composition herein, which is inserted into the lens case or other container in a manner so as to surround and be proximate to the lens or other ocular device stored therein. In some embodiments the treated cloth may be used as a wipe to clean the lenses or other devices to be placed into or on the eye. As part of this cleaning process, some of the antimicrobial composition will be deposited on the lenses or other devices and will provide a barrier against infection from other microorganisms that may be encountered on the surface of the eye or from other sources of contamination that may come into contact with the eye. In some embodiments the pad to enclose the contact lens within the case is treated with the organosilane and further enhanced with a dating system to limit duration of usage. In some embodiments, the concentration of organosilane is less than about 0.1 percent by weight. In some embodiments, the concentration of the organosilane is in the range of from about 0.1 to about 1.0 percent by weight. In some embodiments, the concentration of the organosilane is in the range of from about 1.0 to about 5.0 percent by weight and percent by weight. In some embodiments the concentration of the organosilane is greater than about 5.0 percent by weight.

In some embodiments, the carrier is a compound that includes buffers; sodium chloride; potassium; sugars including disaccharides, such as lactose, monosaccharides, such as dextrose and glucose, and polyols such as mannitol; surfactants; enhancers; saline; and water. Buffers may include boric acid or sodium borate to maintain the pH of the composition in the range of from about 7 to about 8. In some embodiments, when a gel or ointment is desired, the organosilane is mixed with a carrier that may include isotonic saline, in the range of from about 1 to about 2% t polyvinyl alcohol, 1% alpha-methylcellulose, a mixture of white petrolatum-mineral oil ointment or another properly constituted ophthalmic gel, ointment, mineral oil, lanolin or petrolatum.

In some embodiments, the composition also includes an enzyme of bacterial origin, preferably from a Bacillus or Actinomyces, or from fungal sources or genetically engineered from non-alkaline cellulases by modifying the protein to function in an alkaline pH. In some embodiments, the enzyme is a proteolytic keratinase or a protein hydrolase. In some embodiments, the enzyme is an enzyme acting upon a substrate comprising N-acyl homoserine lactone. In some embodiments, the enzyme is an alginate lyase. In some embodiments the enzyme is a cellulose such as carboxymethyl cellulose or a gluconase. In some embodiments, the enzyme is a glycoside hydrolase such as DispersinB. In some embodiments, the enzyme is an amylase or a protease. In some embodiments, the enzyme is Deoxyribonuclease (DNase I.)

Disclosed is a method of providing a non-toxic antimicrobial treatment to inhibit, remove and destroy a biofilm, the method comprising the steps of applying and adhering an antimicrobial composition that includes an organosilane to the cornea by liquid drops or gel or ointment. The antimicrobial composition penetrates the biofilm in some embodiments aided by the use of enzymes and accompanied by other antibiotic and or antifungal compounds designed to destroy intractable colonies together with any disbursed planktonic pathogens. Disclosed is a method of treating an eye infection, the method comprising steps of topically applying the antimicrobial composition containing an organosilane to an infected cornea; thereby penetrating and killing infectious pathogens and biofilms.

In some embodiments for treatment of ocular related articles, the method further comprises the step of placing the treated ocular related article in close proximity to an area of microbial colonization. Disclosed is a method of providing a non-toxic antimicrobial treatment to a container or case for ocular lenses or devices, the method comprising the steps of applying a liquid composition containing an organosilane to the interior surfaces of the case or container by spray, brushing, dipping or other method of application.

The foregoing and other features and advantages of the present invention will be apparent from the following more detailed description of the particular embodiments of the invention.

### BRIEF DESCRIPTION OF THE DRAWINGS

Some of the embodiments will be described in detail, with reference to the following figures, wherein like designations denote like members:

FIG. 1 is a schematic diagram showing a general chemical structure of an organosilane molecule according to the invention;

FIG, 2 is a schematic diagram showing a general chemical structure of an organosilane molecule: 3-(trihydroxysilyl)propyldimethyloctadecyl ammonium chloride according to the invention;

FIG. 3 is a schematic representation showing organosilane molecules adhered to a substrate in the presence of microbial cells according to the invention;

FIG. 4 is a schematic representation of a delivery system for an antimicrobial composition according to the invention;

FIG. 5 is a schematic representation of a delivery system for an antimicrobial composition on a substrate according to the invention;

FIG. 6 is a schematic representation of a delivery system for an antimicrobial composition using an aging indicator according to the invention;

FIG. 7 is a diagram of a method 200 of treating and preventing an infection and/or infectious disease on a substrate according to the invention;

FIG. 8 is a diagram of a method 300 of treating and preventing an infection and/or infectious disease on a substrate according to the invention;

FIG. 9 is a diagram of a method 400 of treating and preventing an infection on a biological substrate according to the invention; and

FIG. 10 is a diagram of a method 500 of treating and preventing an infection on a biological substrate according to the invention.

### DETAILED DESCRIPTION OF EMBODIMENTS OF THE INVENTION

A detailed description of the hereinafter described embodiments of the disclosed apparatus and method are presented by way of example and not meant to be limiting. Although certain embodiments are shown and described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure.

As a preface to the detailed description, it should be noted that, as used in this specification and the appended claims, the singular forms “a”, “an” and “the” include plural referents, unless the context clearly dictates otherwise. Some general definitions are provided for the terms used herein. “Organosilane” means a compound of the family of compounds comprising the elements of silicon, oxygen, and carbon with a C—Si covalent bond and a nitrogen atom in a quaternary ammonium configuration. “Organosilane” also includes any quaternary ammonium salt of an organosilane. “Microbial cell” and “microbe” are used interchangeable and are understood to mean any single-celled planktonic organism. “Biofilms” are multicellular communities usually held together by an extracellular polymeric substance (EPS), ranging from capsular material to cell lysate. In a structure that imposes diffusion limits, environmental microgradients arise to which individual bacteria adapt their physiologies, resulting in the gamut of physiological diversity. Additionally, the proximity of cells within the biofilm creates the opportunity for coordinated behaviors through cellcell communication using diffusible signals, the most well documented being quorum sensing (QS). The cells growing in a EPS biofilm are physiologically distinct from planktonic cells, which, by contrast, are single-cells that may float or swim in a liquid medium. Microparticle may also refer to a solid compound comprising the particle that is, itself, coated with the organosilane for purposes of becoming imbedded in the EPS of a-mature biofilm.

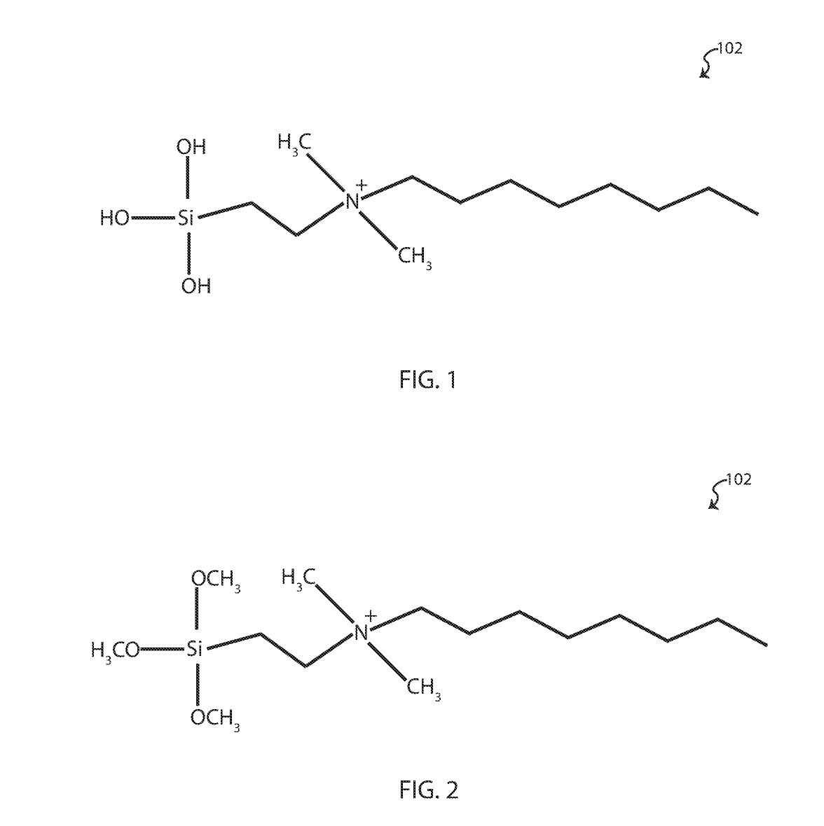

FIGS. 1-6 show an antimicrobial composition 100. Composition 100 is an organosilane 102 in combination with other compounds in a mixture chosen according to the intended application of composition 100.

The antimicrobial action of composition 100 is provided by the organosilane compound. An organosilane is a molecule comprised of a silicone atom covalently bonded to carbon. Organosilanes in general may be amphiphilic, having both water-soluble and lipid soluble components. Organosilane 102 has a hydrophilic “cap” having a silicon-tri-hydroxy “head,” and a hydrophobic “tail” comprising an eighteen or twenty-atom linear carbon chain. The head and tail are joined at a nitrogen atom bonded with two additional methyl groups to create a (cationic) quaternary ammonium group. The methoxy or hydroxy head groups facilitate enzymatically or chemically binding the organosilane to a surface 140. Surface 140 includes the non-limiting examples of biological surfaces, such as corena or mucosa, or non-biological surfaces—whether porous or non-porous—such as a lens or lens case, for example. The hydrophilic quaternary ammonium group, in particular the positive charge of the nitrogen atom, allows for ionic attraction between the negatively-charged cell wall membranes of bacteria and fungi. Microbial cells having a negative ionic charge are drawn to the organosilane electrostatically by the cationic quaternary ammonium groups of the organosilane. Amphiphilic quaternary ammonium compounds, including but not limited to organosilane 102, effect microbial killing by the cationic N+ atom ionically is attracted to negatively charged sites on lipopolysaccharides and constituent proteins of the bacterial cell wall causing perturbation and cell wall weakening with leakage. The carbon chains of the organosilane in proximity to the microbial cell wall engage and may then penetrate the weakened cell wall, destroying the microbe. This cell killing mechanism is advantageous for several reasons. Organosilane 102 is not altered or consumed by its interaction with the targeted cell. Residual organosilane remains covalently bound to the treated substrate-eliminating the need for frequent regular re-application and-minimizing release of large amounts of a frequently-applied compound into the environment.

In various embodiments of the invention, other compounds are added to composition 100. For some embodiments wherein composition 100 is used in a biological system, a carrier, 103 is added. In some embodiments, a carrier is added which may include surfactants, buffers, and/or sodium chloride with water to form aqueous solutions. In some embodiments, a gel or ointment formulation contains a carrier in a hydrophilic base prepared from compounds such as lanolin, mineral oil or polymers. For some embodiments wherein composition 100 is used to kill microorganisms in a biofilm, whether in a biologic or non-biologic environment, composition 100 further comprises a cellulase enzyme. In some embodiments, composition 100 further comprises other enzymes or compounds to interfere with quorum sensing utilized by microorganisms growing in a biofilm. In some embodiments, composition 100 comprises an agent to enhance viscosity. In some embodiments, composition 100 comprises an agent to promote trans-epithelial delivery of un-bound composition 100 through the cornea or mucosal surfaces. The manner and method of mixing these and other ingredients is well known to those skilled in the state of the art.

In some embodiments, composition 100 further comprises nutrients such as, phosphate, sugars, proteins, oxygen and nitrogen. Sugars include monosaccharides, disaccharides and polyols. Monosaccharides are particularly useful. The addition of nutrients effectively feeds the biofilm promoting delay in persister cell formation leading to dormancy and encourages persister activation thus making the active microbe easier to kill by an organosilane that benefits from a strong negative charge on the microbial cell wall. Nutrients appear to induce stress which aids in accomplishing this reversal of dormancy.

Referring to the drawings, FIG. 1 and FIG. 2 each depict an organosilane 102. These non-limiting examples show the fundamental structure of two organosilanes 102 with antimicrobial activity. Composition 100, in some embodiments, may comprise an organosilane 102 with alternative molecular structures. Common to organosilanes 102, however are a silyl “head,” a quaternary ammonium group, and an aliphatic hydrocarbon “tail.” Embodiments of composition 100 comprise organosilane 102 and, in some embodiments, additional structural and functional components that complement one another to add functionality and performance to composition 100, the structure and function of which will be described in greater detail herein.

In the example embodiment shown in FIG. 1, organosilane 102 is a 3-hydroxysilyl organosilane. The silyl “head” of the molecule is shown to the left of the figure, comprising three hydroxyl groups which, in some embodiments, are reacted to covalently bond with a biological or non-biological surface. The quaternary ammonium group is also shown, connecting the silyl “head” with the aliphatic hydrocarbon “tail.” In the example embodiment shown in FIG. 2, organosilane 102 is 3-(trihydroxysilyl) propyl dimethyl octadecyl ammonium molecule. In some embodiments, organosilane 102 is a 3-(trimethoxysilyl) propyl dimethyl octadecyl ammonium molecule, such as 3-(trimethoxysilyltrihydroxysilyl) ammonium chloride, hydrolyzed to the Tri-hydroxy form but without methanol. Physical killing of microbial cells 135 occurs by ionic pertubation of the microbial cell wall and engagement with the organosilane carbon “tails” with penetration and physical disruption of the microbial cell wall and phospholipid cell membrane. Microbial cells 135 are ionically drawn to a treated surface, inter alia a lens case or scleral shield 140 covered with adherent organosilane 102 molecules by the cationic quaternary ammonium groups of organosilane 102.

In some embodiments involving treatment of an eye wherein an invasive microbial infection is present, an anti-inflammatory compound may be desirable as a useful therapeutic adjunct. Invasive microbial infection normally creates an inflammatory response. Inflammation creates swelling, increases pain and/or itching, and, if marked or accompanied by rubbing of the eye, may interfere with healing. Therefore, treatment with a topical or systemic anti-inflammatory compound may be useful. In some embodiments, composition 100 further comprises an anti-inflammatory molecule. Some non-limiting examples of such anti-inflammatory compounds include steroids, such as triamcinolone diacetate, hydrocortisone, beta methasone valerate, and beta methasone diproprionate; non-steroidal anti-inflammatories, resorcinol, and methyl resorcinol

Some antibiotics and enzymes function optimally within a relatively narrow pH range. Accordingly, some embodiments of composition 100 add a buffer to the treating composition at concentration levels sufficient to maintain the pH range required for optimal activity of the components of the composition. The particular buffer is selected based upon the conditions present on the ocular surface 140. Buffers to maintain ambient pH within a desired range include, but are not limited to, boric acid, sodium borate, citrates, sulfonates, carbonates, and phosphates. The preferred buffering compound and concentration of same useful for maintaining a desired pH range are dependent on ambient micro-environmental conditions at the treated area and known to those skilled in the art.

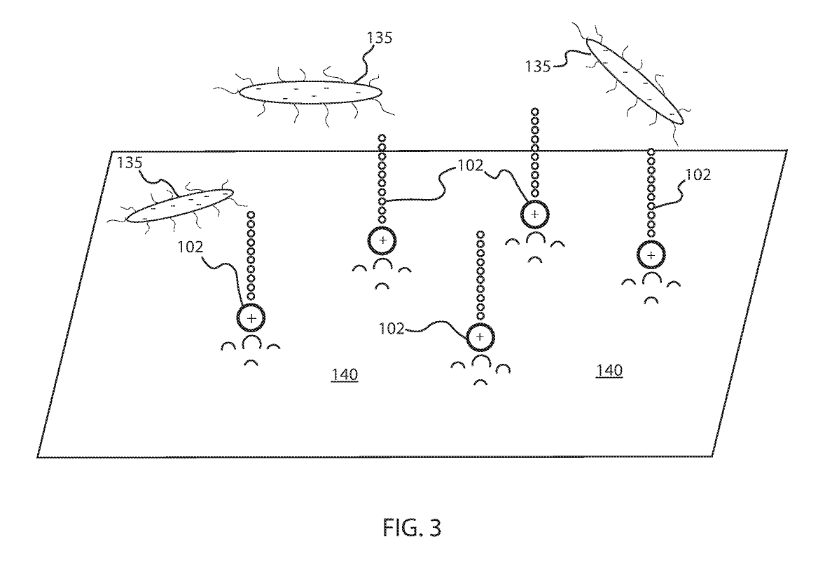

FIG. 3 is a diagram showing organosilane 102 molecules bonded to a lens case surface 140 in the presence of a microbial cell 135. Microbial cells 135 may be bacteria (as shown in FIG. 3), archaebacteria, protists, fungi or a combination thereof. A microbe generally carries a negative net charge on the cell-surface due to constituent membrane lipo-proteins. For example, the cell walls of Gram-positive bacteria contain negatively-charged teichoic acids. The cell membranes of Gram-negative and Gram-positive bacteria (and other microbes) comprise negatively charged phospholipids and lipopolysaccharide molecules. The negatively-charged surfaces of air-borne planktonic” microbes, therefore, are ionically attracted to cationic compounds, such as the quaternary ammonium group-containing organosilane 102 coating surface 140. If the compound, such as organosilane 102, is amphiphilic, the hydrophilic portion of the molecule may traverse both the bacterial cell wall and cytoplasmic membrane, causing cellular lysis and death of microbial cell 135. As a result, the attachment and bonding of composition 100 comprising organosilane 102 to surface 140 results in surface 140 becoming configured to kill microbial cells 135 on contact. Because this surface killing does not disrupt and consume composition 100, frequently repeated application is not required and as a result, cell destruction microbial killing is accomplished without releasing a biocide to the environment.

Composition 100 additionally comprises a carrier 108. Carrier 108, in some embodiments, is a compound that holds the various sub-components of composition 100 in suspension or solution. The specific compound used is chosen based upon the characteristics necessary for the end-use application of composition 100. For example, if composition 100 is to be used to treat a non-biological surface, such as a lens case, carrier 108 may comprises a substance with relatively high volatility, such as ethyl alcohol or isopropyl alcohol or a similar low-molecular weight alcohol, or water. If composition 100 is to be used on a biological surface, such as a cornea, carrier 108 may be an emollient, non-ionic surfactant, viscosity enhancer, salt or sugar-containing solution or other suitable compound. Non-limiting examples include excipients, such as cetyl alcohol, tyloxapol, methyl paraben, white petroleum, propylene glycol, mineral oil, liquid lanolin, cottonseed oil or a polymer liquid-gel. The carrier may, in some embodiments, be employed to form composition 100 into a gel, lotion, ointment, liquid solution, or liquid suspension, according to the intended end-use of composition 100.

The concentration of organosilane 102 by weight of composition 100 is also selected according to the desired end-use of composition 100. In situations where high antimicrobial activity is needed for treating a non-biological surface, higher concentrations of organosilane provide a higher density of adherent organosilane molecules on surface 140. In effect, the “forest” of aliphatic hydrocarbon molecular “tails” is thicker. Additionally, higher organosilane concentrations create a higher cationic charge density, resulting in both stronger electrostatic microbial attractive forces and detergent effects on the microbial phospholipid cell membrane. Because some organosilane molecules become separated from surface 140 with each wiping or cleaning, a higher concentration of organosilane 102 in composition 100, in some embodiments, allows composition 100 to act as a surface antiseptic for a longer period of time. Concentrations of organosilane 102 in composition 100 of up to and over 5% by weight may be used, however, when used in concentrations of over about 3%, polymerization of organosilane 102 within composition 100 prior to application on surface 140 increases through intermolecular cross-linking via—S—O—S—covalent bonds. In applications to biological surfaces, such as a cornea, are to be treated using composition 100, composition shedding through tear shedding and corneal epithelium mitosis requires appropriate re-application of composition 100, in some applications. The risk of developing resistance to an antimicrobial composition, regardless of the reaction mechanism of the compound, theoretically increases with increasing environmental encounters between biofilms and other microbes, and the antimicrobial composition. It is prudent, therefore, to strive to minimize the amount of an antimicrobial composition within the general environment. Accordingly, in the aforementioned and other situations wherein frequent re-application of composition 100 is necessary, lower concentrations of organosilane 102, about 0.1% by weight and lower in composition 100, are useful by lowering the overall amount of organosilane 102 ultimately discharged into the environment. Notwithstanding the theory, it is believed that the risk to the environment and/or causing biofilm mutations by use of these formulations is minimal.

Because in some embodiments, composition is a non-leaching composition that is bound to surface of an ocular related article, such as a gauze pad, felt, cotton or fabric patch, the area may be treated without ever placing or applying the antimicrobial composition directly into the eye. The electrostatic properties of composition 100 comprising an organosilane or and/or additional cationic detergent or other substance may attract and draw nearby microbes to the cationic composition, thereby reducing the concentration of microbes in the area of the eye sought to be protected from microbial colonization and/or infection and possible biofilm formation. In some embodiments, treated article is placed in contact with the eye to treat the infection. In these instances, the positive-negative electrical attraction between the wall of the microbial cells and the formulation in the treated article tends to attract microbes, killing them and maintaining the detritus on the treated article, to be disposed of safely.

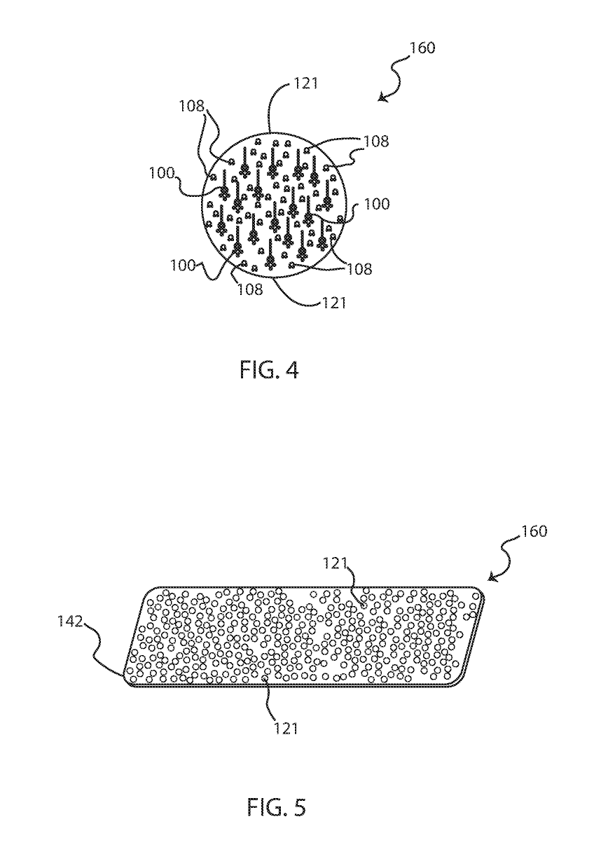

FIGS. 4-6 show a microcapsule 121 encasing composition 100. Microcapsule 121 is one example of delivery system for composition 100. Microcapsule 121, in some embodiments, comprises a material enveloping and containing composition 100. Non-limiting examples of compounds used to form microcapsule 121 include polyvinyl alcohol, cellulose acetate phthalate, gelatin, ethyl cellulose, glyceryl monostearate, bees' wax, stearyl alcohol, and styrene maleic anhydride. Many other compositions of microcapsule 121 are possible, and the exact composition, construction, and manufacture of microcapsule 121 is chosen from the broad range of compositions and manufacturing techniques for microcapsules generally, and which are readily available and known to those skilled in the art. In the example delivery system shown in FIG. 4, liquid composition 100 is encapsulated within microcapsule 121 and thereafter released when microcapsule 121 is broken. Breakage of microcapsule 121 is effected at a chosen time and in a manner specific to the particular use of composition 100. For example, microcapsule 121 may be broken by scratching or abrading the area. In this manner, composition 100 is configured to remain on substrate 140. Because composition 100 becomes active upon breaking of microcapsule 121, the effective useful life of product composition begins.

In some embodiments, as shown in FIG. 5, treated article 142 is used to disinfect or prevent infection of a lens or other article placed on or into the eye. In some embodiments, composition 100 and/or delivery system 160 is applied to an existing biofilm. Composition 100 comprising organosilane 102 with amphiphilic properties penetrates an existing biofilm, bringing the biocidal organosilane 102, along with additional antibiotic and/or antiseptic compounds, in some embodiments, to deeper layers of an existing biofilm, killing microbial cells 135 within the extracellular biofilm matrix and disrupting the biofilm. In some embodiments, composition 100 is applied as an aerosol, other spray, brushed or wiped onto surface of an object used to store ocular objects, such as a lens case. In some embodiments, an eye with existing microbial contamination, with or without an associated biofilm, is treated directly, topically by applying liquid composition 100.

The use of composition 100, as an antimicrobial on a non living surface is prone to deactivation and creation of the very condition that it intends to prevent. This is because cellular debris from killed microbes may adhere to the hydrophilic “tail” of organosilane after death and new approaching microbes can adhere and proliferate on this debris, 135, some embodiments of composition 100 may be self-deactivating. Additionally, biologic exudates such as mucopolysaccharides, inorganic dust and other particulate matter and cellular material from dead microbes may eventually fill and clog the microscopic bed of composition 100, thus forming a favorable local microenvironment for the development of new biofilms. The microscopic bed of composition 100 may then become a biofilm that use of composition 100 is intended to prevent.

In addition to proteolytic keratinases, some embodiments of composition 100 comprise other enzymes. For example, N-acyl homoserine lactone is a bacterially-produced amino sugar acting as a hormone involved in quorum sensing, wherein a population of bacteria limits its growth density and other population-based characteristics, such as gene regulation of enzyme systems and the expression of flagella versus pili. Enzymes acting upon an N-acyl homoserine lactone substrate destroy and substrate and thereby temporarily disrupt bacterial signaling systems in a biofilm, acting as an adjunct to proteolytic keratinases and other components of composition 100, in some embodiments, such disruption may cause the existing biofilms to break apart and interfere with new biofilm formation. . In some embodiments, the enzyme is an alginate lyase. In some embodiments the enzyme is a cellulase such as carboxymethyl cellulase or a gluconase In some embodiments, the enzyme is a glycoside hydrolase such as DispersinB. In some embodiments, the enzyme is an amylase or a protease. In some embodiments, the enzyme is Deoxyribonuclease (DNase I.)

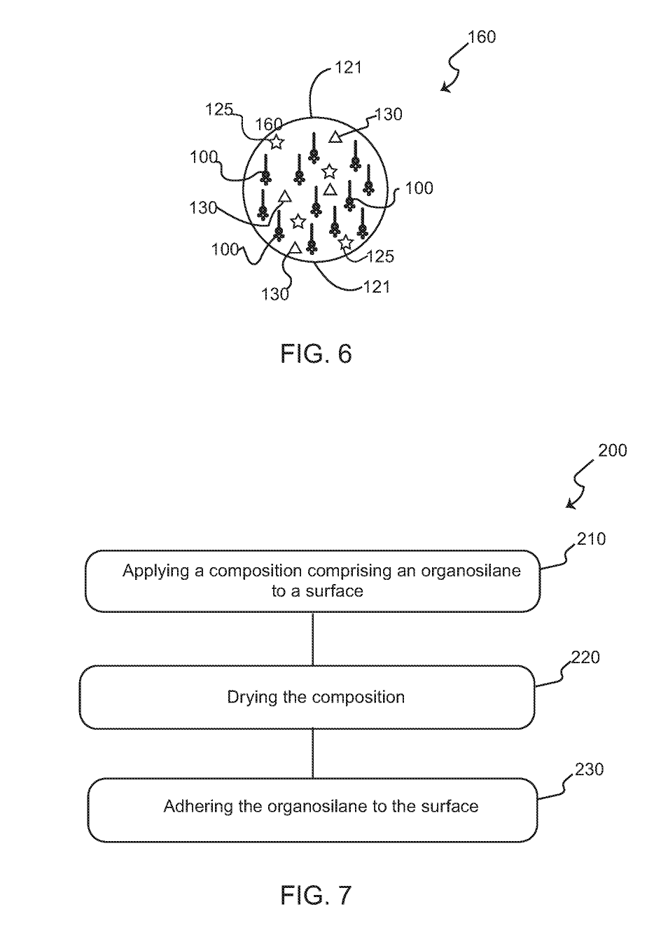

FIG. 6 shows delivery system 160 for composition 100 further comprising aging indicator 125. Aging indicator 125, in some embodiments, is configured to exert, exhibit, or otherwise release a color, fading agent, or time-dependent color that changes color over a predetermined period of time after aging indicator 125 has been activated. Some embodiments of delivery system 160 comprise aging indicator 125 comprising a fading color or time-dependent color that changes color or alters color for a time period matching the useful life of composition 100's biological activity. In other words, some embodiments of the delivery system 160 comprise a time period calculated and configured to match the anticipated life expectancy of composition 100. In this way, the user is able to determine by the color, faded color, or time-dependent color whether composition 100 remains biologically active or has expired. Once expired, the user is on notice that composition 100 on is no longer biologically active and that consideration should be given to discarding article 142.

FIG. 7 shows a method 200 of treating infection and/or infectious disease and/or providing long-lasting antimicrobial properties to a substrate that is used to clean ocular surfaces or reduce infectious pathogens by proximity. Method 200 comprises an applying step 210 and an adhering step 230. Step 210 of method 200 comprises applying composition 100 comprising an organosilane to a substrate. Step 210, in some embodiments, includes applying composition 100 to a article. Step 220 of method 200 comprises adhering the organosilane to the substrate In some embodiments, adhering step 230 comprises formation of covalent bonds between the organosilane and the surface. In some embodiments, adhering step 220 comprises adsorption onto a non-porous substrate or into a porous surface. In some embodiments, adhering step 220 comprises an electrostatic interaction between the organosilane and the substrate, such as formation of ionic chemical bonds, for example. In some embodiments, adhering step 220 comprises addition of an additional compound, such as a catalyst, to accelerate reaction of the organosilane with the substrate. In some embodiments, other bonding agents and/or techniques are employed to facilitate bonding of composition 100 with the material of the treated article.

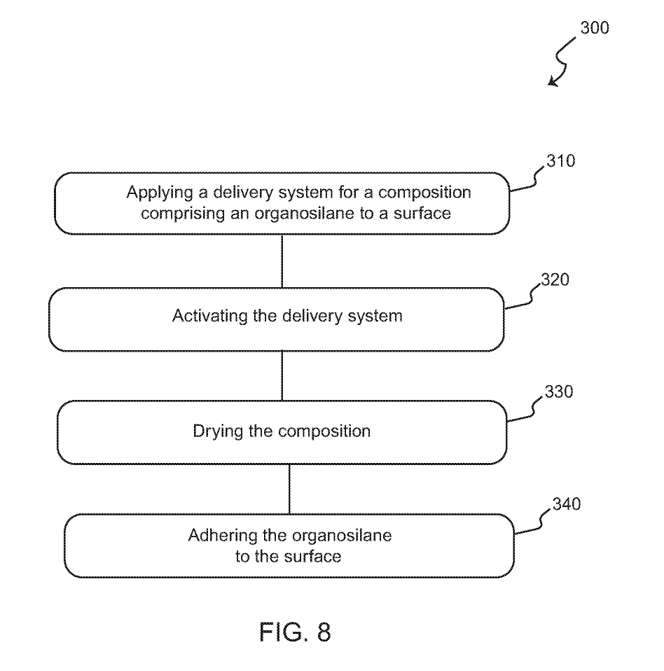

In some embodiments as shown in FIG. 8, a method 300 may include applying step 310. Applying step 310 comprises integrating the delivery system, containing a composition comprising an organosilane, into part or all of the material composition of the treated article during manufacture. For example, the pad for insertion into a lens case or a patch to be placed over or on the eye, in some embodiments, is manufactured to contain a quantity of the composition upon and intermingled within the fibers throughout the pad or patch. Step 320 of method 300 comprises activating the delivery system. In some embodiments, activating step 320 comprises removing the treated article from its packaging. Bonding of the organosilane to the article may be by covalent bonding, ionic bonding, electrostatic bonding, or other interaction between the organosilane and the surface material.

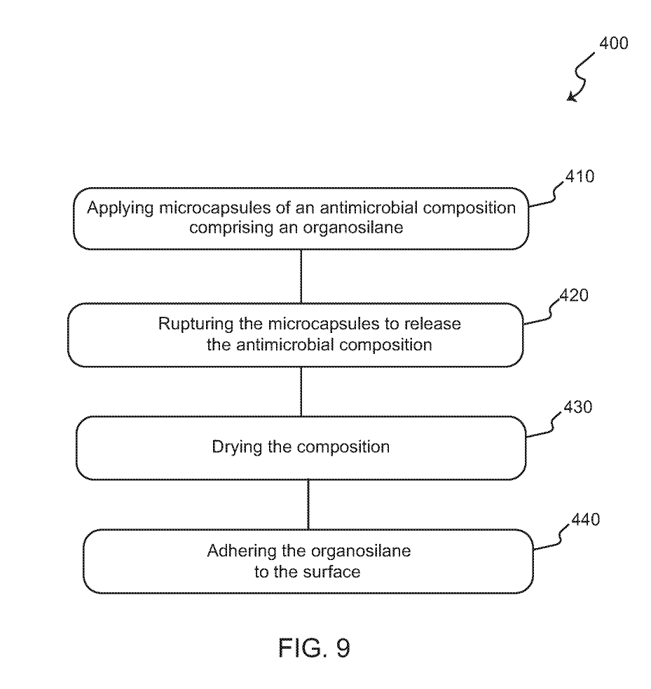

In some embodiments as shown in FIG. 9, a method 400 including applying the antimicrobial composition to the surface of a substrate wherein the antimicrobial product comprising organosilane is both coated on and embedded in the microcapsules 410; rupturing the microcapsules to release the antimicrobial composition 420; and adhering the antimicrobial composition the surface of the substrate.

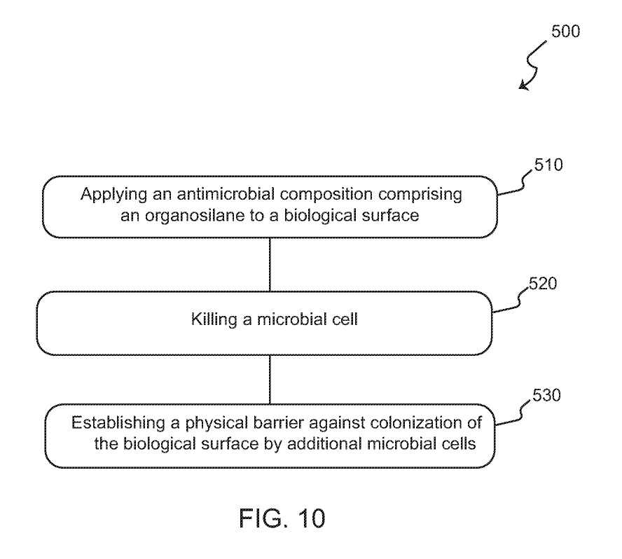

FIG. 10 shows a method 500 of treating and preventing the spread of an infection on and in an ocular area. Method 500 comprises an applying step 510, a killing step 520, and an establishing step 530. Applying step 510, in some embodiments, comprises applying an antimicrobial composition comprising an organosilane to an ocular area. The biological surface, in some embodiments, is a site of invasive ocular infection and may include a high density of bacterial, fungi, and/or other microorganisms. Killing step 520 comprises the killing of microbial cells via the reaction mechanism(s) of the antimicrobial composition.

Exceptional results can be obtained with organosilane compounds for treatment of human and animal eye infection, creating antiseptic coatings for tissues, and methods of using the same disclosed in this description of several embodiments of the invention. The disclosed composition provides a durable treatment of a biological or non-biologic surface, minimizes leaching of antimicrobial into the environment, minimizes opportunities for development of microbial resistance due to its combined physical and electrostatic mechanisms of action, is safe and effective in treating resistant invasive infections of the eye and surrounding tissues, and may be applied directly to articles such as lens cases and containers.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

What is claimed is:

1. An antimicrobial composition comprising:
3-(trihydroxysilyl)propyldimethyloctadecyl ammonium chloride and a sugar.

2. The antimicrobial composition of claim 1, wherein the sugar is a disaccharide.

3. The antimicrobial composition of claim 1, wherein the sugar is a monosaccharide.

4. The antimicrobial composition of claim 1, wherein the antimicrobial composition is a liquid.

5. The antimicrobial composition of claim 1, wherein the antimicrobial composition is a gel, foam or ointment.

6. The antimicrobial composition of claim 1, wherein the 3-(trihydroxysilyl)propyldimethyloctadecyl ammonium chloride is hydrolyzed 3-(trimethoxysilyltrihydroxysilyl) ammonium chloride.

7. The antimicrobial composition of claim 1, wherein the concentration of the organosilane is less than about 0.1 percent by weight.

8. The antimicrobial composition of claim 1, wherein the concentration of the organosilane is in the range of from about 0.1 percent to about 1.0 percent by weight.

9. The antimicrobial composition of claim 1, wherein the concentration of the organosilane is greater than about 1.0 percent by weight.

10. The antimicrobial composition of claim 1, wherein the concentration of the organosilane is greater than 5.0 percent by weight.

11. The antimicrobial composition of claim 1, wherein the carrier is a compound selected from the group of mineral oil, liquid lanolin, white petrolatum, isotonic saline, polyvinyl alcohol, alpha-methylcellulose, a polymer liquid-gel formulation and/or mixtures thereof.

12. The antimicrobial composition of claim 1, further comprising an enzyme.

13. The antimicrobial composition of claim 12, wherein the enzyme is a proteolytic keratinase enzyme.

14. The antimicrobial composition of claim 12, wherein the enzyme is N-acyl homoserine lactone.

15. The antimicrobial composition of claim 12, wherein the enzyme is an amylase.

16. The antimicrobial compisition of claim 12, wherein the enzyme is a cellulase.

17. The antimicrobial composition of claim 12, wherein the enzyme is an alginate lyase.

18. The antimicrobial composition of claim 12, wherein the enzyme is is a glycoside hydrolase, such as DispersinB.

19. The antimicrobial composition of claim 12, wherein the enzyme is a Dnase I, (Deoxyribonuclease.)

20. The antimicrobial composition of claim 12, wherein the enzyme is a protease.

21. The antimicrobial composition of claim 1, further comprising an alcohol.

22. The antimicrobial composition of claim 1, further comprising a buffer to maintain a desired pH level.

23. The antimicrobial composition of claim 22, wherein the buffer is selected from the group consisting of citrate, sulfonate, carbonate, phosphate and/or mixtures thereof.

24. A method of antimicrobial treatment of a surface of an ocular article comprising:
applying a composition comprising 3-(trihydroxysilyl)propyldimethyloctadecyl ammonium chloride and a sugar directly to the surface of the ocular article, whereby the antimicrobial composition adheres to the surface of the ocular article.

25. The method of claim 24, further comprising a biofilm on the surface, whereby the antimicrobial composition penetrates the biofilm.

26. The method of claim 24, wherein the antimicrobial composition is applied to the surface during manufacture of the ocular article.

27. The method of claim 26, further comprising washing and drying the ocular article and reapplying the composition directly to the surface of the ocular article.

28. A method of antimicrobial treatment of an eye comprising applying an antimicrobial composition comprising 3-(trihydroxysilyl)propyldimethyloctadecyl ammonium chloride and a sugar directly to a surface of the eye; whereby microbial cells on the surface of the eye are killed and a physical barrier against colonization by additional microbial cells is established.
